(19) United States
(12) Patent Application Publication
(10) Pub. No.: US 2021/0123864 A1
(43) Pub. Date: Apr. 29, 2021

(54) FIRE DETECTION APPARATUS AND METHOD USING LIGHT SPECTRUM ANALYSIS

(71) Applicant: ELECTRONICS AND TELECOMMUNICATIONS RESEARCH INSTITUTE, Daejeon (KR)

(72) Inventors: Hoe Sung YANG, Daejeon (KR); So Yung PARK, Daejeon (KR); Kang Bok LEE, Daejeon (KR); Kyu Won HAN, Daejeon (KR)

(21) Appl. No.: 17/069,137

(22) Filed: Oct. 13, 2020

(30) Foreign Application Priority Data
Oct. 25, 2019 (KR) 10-2019-0133873
Jul. 3, 2020 (KR) 10-2020-0082431

(51) Int. Cl. G01N 21/63; G08B 29/18; G08B 17/103; G01N 21/31; G01N 33/00

(52) U.S. Cl. CPC G01N 21/63; G08B 29/185; G01N 33/0036; G01N 21/31; G08B 17/103

(57) ABSTRACT

Provided are a fire detection apparatus and method for analyzing a spectral distribution of secondary light generated as primary light is scattered or transmitted through smoke particles to distinguish between fire smoke generated due to an actual fire and living smoke generated in daily life, thereby reducing non-fire alarms. When smoke enters the inside of the fire detection apparatus (100) due to a fire, secondary light (150) scattered or transmitted through smoke particles (140) is incident on the light receiver (120). Upon receiving the secondary light (150), the light receiver (120) outputs a spectrum (170) of the secondary light (150). The fire identification unit (160) receives and analyzes the spectrum (170) of the secondary light (150) and identifies whether the smoke particles (140) are particles of living smoke or particles of fire smoke.

### CROSS-REFERENCE TO RELATED APPLICATIONS

This application claims priority to and the benefit of Korean Patent Application No. 10-2019-0133873, filed on Oct. 25, 2019 and Korean Patent Application No. 10-2020-0082431, filed on Jul. 3, 2020, the disclosures of which are incorporated herein by reference in their entirety.

### BACKGROUND

### 1. Field of the Invention

The present disclosure relates to fire detection technology, and more particularly, to a fire detection apparatus and method for reducing non-fire alarms by distinguishing between the fire smoke generated from an actual fire and the non-fire smoke generated in daily life (hereinafter, referred to as living smoke).

### 2. Description of Related Art

Fire detectors are devices that sense heat or smoke generated during a fire to detect the fire early and are fire-fighting devices for fire detection that automatically detect a fire and sound a fire alarm when the fire occurs. Fire detectors include heat detectors, smoke detectors, heat and smoke detectors, flame detectors, and the like. The heat detectors are categorized into a differential heat detector that detects a fire when a temperature increases sharply, a fixed temperature heat detector that detects a fire when a temperature increases above a set temperature, and a compensation heat detector that may be used as both the differential heat detector and the fixed temperature heat detector, and may be classified into a spot type heat detector and a distribution type heat detector according to a range of detection. The smoke detectors operate upon detecting smoke generated during a fire. The smoke detectors include an ionization type smoke detector that uses a change in an ion current when smoke enters a sensing part and a photoelectric smoke detector that uses a change in the amount of light incident on a photoelectric element when smoke enters a sensing part. The heat and smoke detectors have a function of compensation heat sensing and a function of photoelectric smoke sensing to simultaneously sense both heat and smoke. The flame detectors operate when the amount of change of flame in a fire is greater than a certain level and may operate according to a change in the amount of light received by a light-receiving element due to a flame at a position. The flame detectors may be classified into an ultraviolet flame detector, an infrared flame detector, a UV-infrared flame detector, and a hybrid flame detector.

Generally, in order to detect a fire in a house, a building or the like, such a fire detector is installed by attaching a base thereof to a ceiling, a wall or the like and assembling a detector, which consists of elements in a circuit configuration, on the base. When a fire occurs, the fire detector senses flame, smoke, a temperature, etc., and transmits a signal to the outside to sound an alarm.

FIG. 1 is a schematic diagram for describing the principle of a general photoelectric fire detector 10 that uses a change in the amount of light incident on a photoelectric element when smoke enters a sensing part. The general photoelectric fire detector 10 includes a light emitter 11 emitting infrared light of about 900 nm and a light receiver 12. And it is configured that, when light 13 emitted from the light emitter 11 is incident on the light receiver 12, the light receiver 12 reacts to the light 13. Because the light receiver 12 is arranged to be misaligned with a path of the light 13 emitted from the light emitter 11, the light 13 of the light emitter 11 is not incident on the light receiver 12 in a normal environment in which no smoke is generated.

FIG. 2 is a diagram for describing a smoke detection process performed when smoke enters the general photoelectric fire detector 10 of FIG. 1. As described above, because the light emitter 11 and the light receiver 12 are arranged to be misaligned with each other, the light 13 is not incident on the light receiver 12 in a normal environment. However, when smoke enters the photoelectric fire detector 10, a part of the light 13 emitted from the light emitter 11 is scattered by smoke particles 14 and thus scattered light 15 is incident on the light receiver 12. The light receiver 12 is designed to be simply turned on or off or to output a logic high or low signal according to whether the scattered light 15 is detected.

However, because the general photoelectric fire detector 10 operates only in response to the scattered light 15 generated due to the smoke particles 14 entering the inside thereof including the light emitter 11 and the light receiver 12, the general photoelectric fire detector 10 may operate in response to cigarette smoke, cooking smoke, dust, etc., thereby causing frequent issuance of non-fire alarms (non-fire alerts).

### SUMMARY OF THE INVENTION

As described above, the present disclosure is designed to solve a problem that heat from daily life such as heat from sunlight, a halogen lamp, a heater, etc. or living smoke such as cigarette smoke, cooking smoke, fine dust, etc. in a normal environment is frequently erroneously detected as a fire and a non-fire alarm is issued by a fire detector. Accordingly, the present disclosure is directed to reducing non-fire alarms by distinguishing between fire smoke generated from an actual fire and living smoke generated from daily life.

To this end, a smoke detector that analyzes a spectral distribution of light scattered by smoke particles is used. A light spectrum analysis-based fire detection apparatus and method according to the present disclosure includes a light emitter, a light receiver having a light spectrum detection function, and a fire identification unit for identifying a fire by analyzing a light spectrum.

Specifically, an aspect of the present disclosure includes the following:
emitting light (primary light) to smoke particles through at least one light emitter;
receiving, by a light receiver, secondary light generated as the primary light emitted from the light emitter is scattered or transmitted through the smoke particles and detecting a light spectrum from the secondary light;
building and using a large amount of light spectrum database (DB) to distinguish between a fire and a non-fire by identifying smoke generated due to a fire and non-fire smoke generated in daily life; and
using an artificial intelligence learning method to analyze the light spectrum.

The concept of the present disclosure described above will be more apparent through embodiments described in detail below in conjunction to the accompanying drawings.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a diagram for describing the principle of a general photoelectric fire detector;

FIG. 2 is a schematic diagram illustrating an operating process of the photoelectric fire detector of FIG. 1 when smoke enters therein;

FIGS. 3 and 4 are schematic configuration diagrams of a fire detection apparatus based on light spectrum analysis according to an embodiment of the present disclosure;

FIG. 5 is a diagram for describing an operation of the present disclosure when smoke particles of daily life enter a fire detection apparatus based on a light spectrum analysis according to an embodiment of the present disclosure;

FIG. 6 is a diagram for describing an operation of the present disclosure when smoke particles of a fire enter a fire detection apparatus based on a light spectrum analysis according to an embodiment of the present disclosure; and

FIG. 7 is a flowchart of a fire detection method based on light spectrum analysis according to an embodiment of the present disclosure.

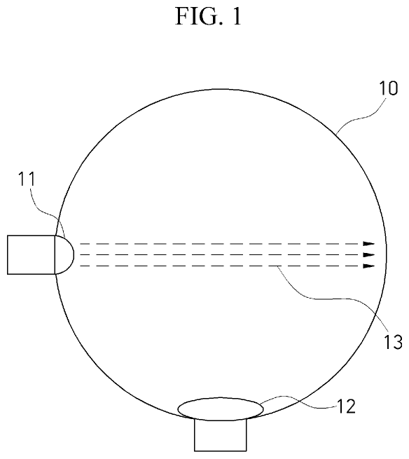

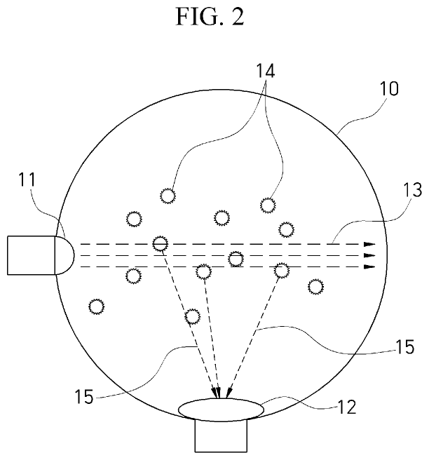

### DETAILED DESCRIPTION OF EXEMPLARY EMBODIMENTS

Advantages and features of the present disclosure and methods of achieving them will be apparent from the following description of embodiments in conjunction with the accompanying drawings. The present disclosure is not limited to embodiments set forth herein and may be embodied in many different forms. These embodiments are provided so that this disclosure will be thorough and complete and will fully convey the concept of the present disclosure to those of ordinary skill in the art, and the scope of the present disclosure should be defined by the claims.

The terminology used herein is for the purpose of describing particular embodiments only and is not intended to be limiting of the present disclosure. As used herein, singular forms are intended to include plural forms unless the context clearly indicates otherwise. As used herein, the terms “comprise” or “comprising” specify the presence of stated components, steps, operations and/or elements but do not preclude the presence or addition of one or more other components, steps, operations and/or elements.

Hereinafter, embodiments of the present disclosure will be described in detail with reference to the accompanying drawings. In the following description of embodiments, well-known functions or configurations are not described in detail when it is determined that they would obscure the present disclosure due to unnecessary detail.

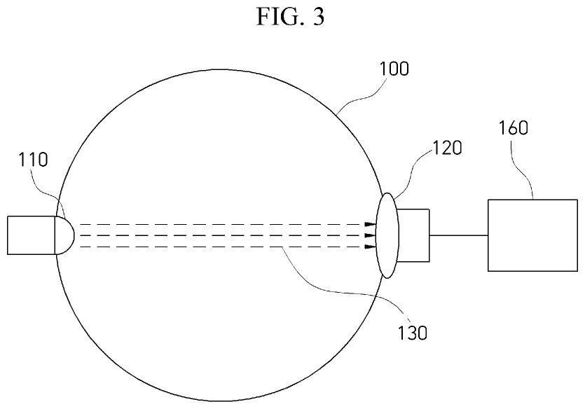

FIG. 3 is a schematic configuration diagram for describing a fire detection apparatus and method using light spectrum analysis according to an embodiment of the present disclosure, in which a state in which smoke particles do not enter a fire detection apparatus 100 is schematically illustrated. The fire detection apparatus 100 according to the present disclosure includes one or more light emitters 110, a light receiver 120 for detection of a light spectrum, and a fire identification unit 160. When no smoke particles enter the fire detection apparatus 100, primary light 130 emitted from the light emitter 110 is directly detected by the light receiver 120. Therefore, the fire identification unit 160 receives the same spectrum as that of the primary light 130 emitted from the light emitter 110, analyzes the received spectrum, and determines that a current situation corresponds to a non-fire.

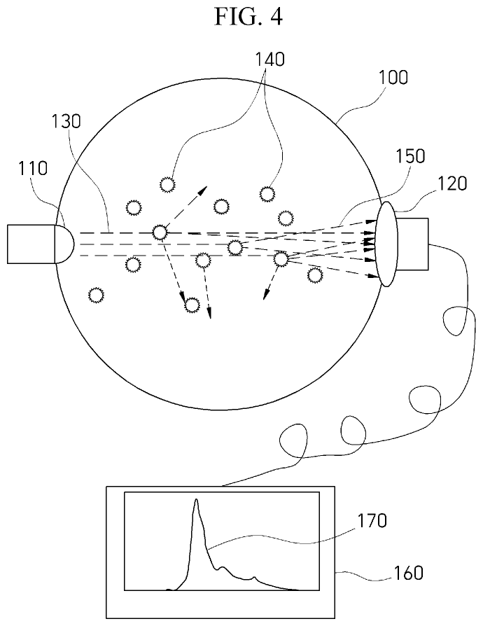

FIG. 4 is a diagram for describing an operation of the present disclosure when smoke particles enter the fire detection apparatus 100 due to a fire.

When smoke enters the fire detection apparatus 100, primary light 130 emitted from the light emitter 110 is scattered or transmitted through smoke particles 140. The light receiver 120 receives light (‘secondary light’) 150 generated as the primary light 130 is scattered or transmitted. The light receiver 120 has a light spectrum detection capability and thus outputs a spectrum 170 of the received secondary light 150. The fire identification unit 160 receives and analyzes the spectrum 170 of the secondary light 150 output from the light receiver 120 and identifies whether smoke particles entering the fire detection apparatus 100 are particles of living smoke or particles of fire smoke, thereby distinguishing between a fire or a non-fire.

As described above, the principle of the present disclosure uses the fact that a spectrum of a wavelength of secondary light varies according to whether living smoke or smoke of an actual fire enters. For example, the size of the wavelength of the secondary light generated due to scattering or transmitting of light through smoke particles may decrease or a wavelength shift may occur according to whether living smoke or smoke of an actual fire enters. It is the principle of the present disclosure to analyze a spectrum of the secondary light to distinguish between a fire and a non-fire.

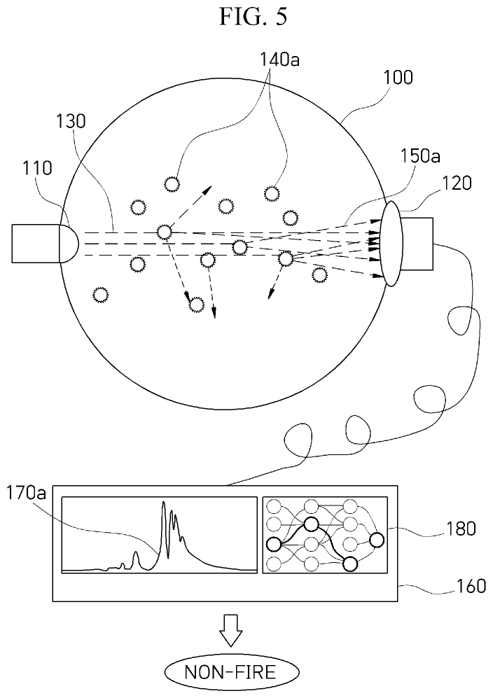

FIG. 5 is a diagram for describing identification of a non-fire by applying a smoke identification algorithm 180 installed in the fire identification unit 160 to a spectrum 170a output when smoke particles 140a from daily life enter the fire detection apparatus 100 of FIG. 4 and secondary light 150a generated due to scattering or transmitting of light through the smoke particles 140a of the living smoke is received by the light receiver 120.

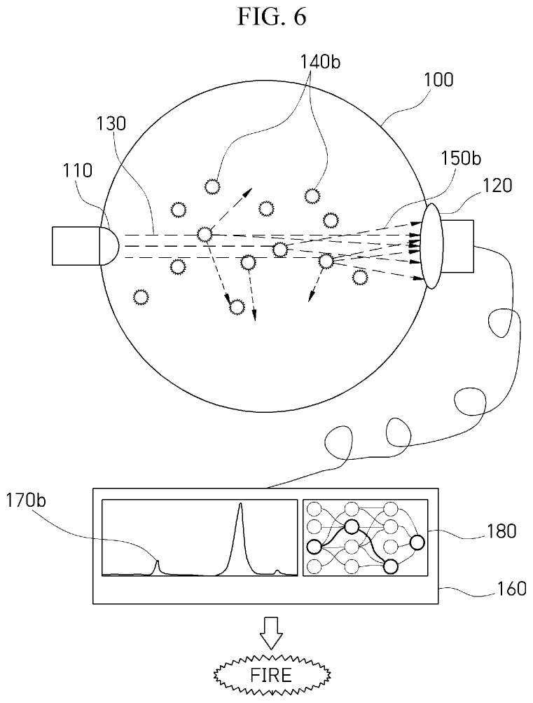

FIG. 6 is a diagram for describing identification of a fire by applying the smoke identification algorithm 180 installed in the fire identification unit 160 to a spectrum 170b output when smoke particles 140b of an actual fire enter the fire detection apparatus 100 of FIG. 4 and secondary light 150b generated due to scattering or transmitting of light through the smoke particles 140b of the actual fire is received by the light receiver 120.

Referring to FIGS. 5 and 6, a fire detection apparatus and method according to the present disclosure uses the fact that a light spectrum of the secondary light 150a when the smoke particles 140a of daily life enter the fire detection apparatus 100 and a light spectrum of the secondary light 150b when the smoke particles 140b of the actual fire enter the fire detection apparatus 100 are different. As is well known, the spectrum of light consists of ultraviolet light of about 400 nm or less, visible light of about 400 to 700 nm or infrared light of about 700 nm or more according to a wavelength. Here, the visible light can be seen by the human eye but the ultraviolet light and the infrared light are almost invisible to the human eye.

Referring to FIGS. 3 to 5, the light emitter 110 may be configured to generate primary light using one light-emitting element but may be also configured to generate light having a plurality of desired wavelength bands using a plurality of light-emitting elements. When the plurality of light-emitting elements are used as in the latter, all the light-emitting elements may be continuously and simultaneously driven or may be pulse-driven sequentially, at the same time, or randomly.

When the secondary light 150a or 150b generated as a part of the primary light 130 emitted from the light emitter 110 is scattered or transmitted through the smoke particles 140a or 140b is incident on the light receiver 120, the light receiver 120 outputs the spectrum 170a or 170b having a pattern in which an amplitude varies according to a wavelength band.

The light receiver 120 may be embodied as a spectrometer. One or more light receivers 120 may be used. When a plurality of light receivers 120 are used, a spectrum of a desired band may be detected using a plurality of light-receiving elements configured to detect different wavelength bands or the difference between secondary light rays received at different positions may be detected using a plurality of light-receiving elements configured to measure the same wavelength band. When the plurality of light-receiving elements for detection of a light spectrum are used, all the light-receiving elements may be continuously and simultaneously driven or may be pulse-driven sequentially or at the same time or randomly.

Next, the fire identification unit 160 distinguishes between fire smoke and living smoke by analyzing a spectrum of each wavelength band of light (secondary light) detected by the light receiver 120 using the smoke identification algorithm 180 to identify fire smoke on the basis of a result of the analyzing.

To distinguish between fire smoke and living smoke using the smoke identification algorithm 180, the fire identification unit 160 may refer to a database built with secondary-light spectrum data of various types of smoke that have been previously investigated. Secondary-light spectrum data according to various fire smoke particles may be obtained according to a cause or aspect of a fire or the like, and similarly, secondary-light spectrum data according to various living smoke particles may be obtained. The secondary-light spectrum data may be collected and analyzed in advance to build a secondary-light spectrum database of smoke particles. For reference of the secondary-light spectrum database, indexes such as a peak value of the intensity of light for each wavelength or a distribution position and number of peak values of the intensity of light for each wavelength may be used.

Artificial intelligence learning techniques such as deep neural networks may be used for execution of the smoke identification algorithm 180. In this case, a learning model may be built through machine learning such as deep learning using various secondary-light spectra of fire smoke and living smoke as training data, and whether a currently detected light spectrum corresponds to fire smoke or living smoke may be inferred using the learning model.

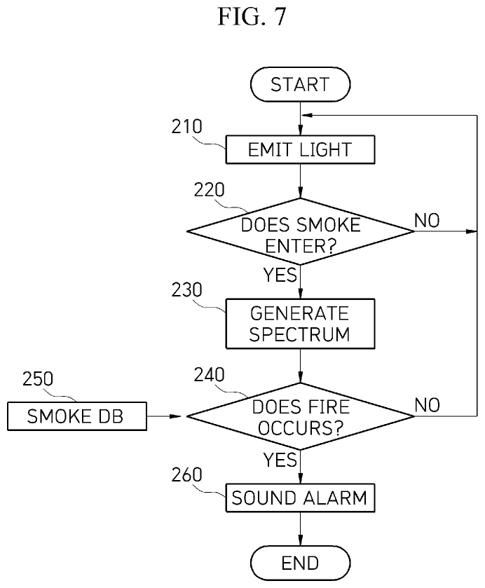

FIG. 7 is a flowchart of a fire detection method based on light spectrum analysis according to an embodiment of the present disclosure.

210: Primary light is emitted from a light source (for example, the light emitter 110).

220, 230: A light spectrum is generated from secondary light generated as the primary light is scattered or transmitted through smoke particles due to introduction of smoke (for example, into the fire detection apparatus 100 of FIG. 3). The generated light spectrum is detected (e.g., by the light receiver 120) as a distribution of each wavelength band having a specific pattern.

240, 260: The detected spectrum is compared with, for example, a wavelength band distribution spectrum according to a type of smoke, which is stored in a secondary-light spectrum DB (smoke DB) 250 for the smoke as described above. Machine learning may be used in this case. Through the comparison of the spectrums, it is determined whether the introduced smoke is fire smoke or living smoke to determine whether a fire has occurred and whether to issue a fire alarm.

Among the components of the present disclosure described above, in particular, the function or process of the fire identification unit 160 may be implemented using hardware components, including at least one of a digital signal processor (DSP), a processor, a controller, an application-specific integrated circuit (IC) (ASIC), a programmable logic device (a field programmable gate array (FPGA) or the like), and other electronic devices or and combinations thereof. Alternatively, the function or process of each component of the present disclosure may be implemented by software alone or in combination with the hardware component elements. The software can be stored in a recording medium.

When the fire detection technology according to the present disclosure for distinguishing between fire smoke and living smoke on the basis of light spectrum analysis is employed, it is effective to reduce non-fire alarms issued by a fire detector operating due to erroneous determination of a non-fire as a fire.

While the present disclosure has been described above in detail with respect to embodiments, it will be understood by those of ordinary skill in the art that the present disclosure can be embodied in many different forms without departing from the technical idea or essential features of the present disclosure. Accordingly, the embodiments set forth herein should be considered only as examples and not for purposes of limitation. The scope of the present disclosure is defined by the following claims rather than the detailed description, and all changes or modifications derivable from the claims and their equivalents should be construed as being included in the technical scope of the present disclosure.

What is claimed is:

1. A fire detection apparatus using a light spectrum analysis, comprising:
a light emitter configured to emit light;
a light receiver configured to receive secondary light generated when the light emitted from the light emitter is scattered or transmitted through smoke particles and to detect a light spectrum having a pattern in which an amplitude varies according to a wavelength band of the received secondary light; and
a fire identification unit configured to distinguish between a fire and a non-fire by analyzing the light spectrum output from the light receiver and identifying whether the smoke particles are particles of living smoke or particles of fire smoke.

2. The fire detection apparatus of claim 1, wherein the wavelength band of the light emitted from the light emitter comprises an ultraviolet band, a visible light band, and an infrared band.

3. The fire detection apparatus of claim 1, wherein the wavelength band of the light emitted from the light emitter comprises at least one of an ultraviolet band, a visible light band, and an infrared band.

4. The fire detection apparatus of claim 1, wherein the light emitter comprises two or more light-emitting elements,
wherein the two or more light-emitting elements are simultaneously driven.

5. The fire detection apparatus of claim 1, wherein the light emitter comprises two or more light-emitting elements,
wherein the two or more light-emitting elements are individually pulse-driven.

6. The fire detection apparatus of claim 1, wherein the light receiver comprises a spectrometer.

7. The fire detection apparatus of claim 1, wherein the light receiver comprises two or more light-receiving elements configured to detect different wavelength bands.

8. The fire detection apparatus of claim 7, wherein the two or more light-receiving elements are simultaneously driven.

9. The fire detection apparatus of claim 7, wherein the two or more light-receiving elements are individually pulse-driven.

10. The fire detection apparatus of claim 1, wherein the light receiver comprises two or more light-receiving elements configured to measure the same wavelength and thus is capable of detecting a difference between secondary light rays which are received at different positions.

11. The fire detection apparatus of claim 10, wherein the two or more light-receiving elements are simultaneously driven.

12. The fire detection apparatus of claim 10, wherein the two or more light-receiving elements are individually pulse-driven.

13. The fire detection apparatus of claim 1, wherein the fire identification unit references a database built with data about various secondary-light spectra of fire smoke and living smoke to distinguish between fire smoke and living smoke.

14. The fire detection apparatus of claim 1, wherein the fire identification unit infers whether the light spectrum detected by the light receiver corresponds to smoke fire or living smoke through a learning model machine-trained with various secondary light spectra of fire smoke and living smoke as training data so as to distinguish between fire smoke and living smoke.

15. A fire detection method using a light spectrum analysis, comprising:
(1) emitting light to smoke particles;
(2) receiving secondary light generated as the emitted light is scattered or transmitted through smoke particles and detecting a light spectrum having a pattern in which an amplitude varies according to a wavelength band of the received secondary light; and
(3) analyzing the detected light spectrum to identify whether the smoke particles are particles of living smoke or particles of fire smoke, thereby distinguishing between a fire and a non-fire.

16. The fire detection method of claim 15, wherein the wavelength band of the light emitted in operation (1) comprises an ultraviolet band, a visible light band, and an infrared band.

17. The fire detection method of claim 15, wherein the wavelength band of the light emitted in operation (1) comprises at least one of an ultraviolet band, a visible light band, and an infrared band.

18. The fire detection method of claim 15, wherein operation (3) comprises referencing a database built with data about various secondary-light spectra of fire smoke and living smoke to distinguish between fire smoke and living smoke.

19. The fire detection method of claim 15, wherein operation (3) comprises inferring whether the light spectrum detected in operation (2) corresponds to smoke fire or living smoke through a learning model machine-trained with various secondary light spectra of fire smoke and living smoke as training data so as to distinguish between fire smoke and living smoke.
